(19) United States
(12) Patent Application Publication
(10) Pub. No.: US 2024/0404633 A1
(43) Pub. Date: Dec. 5, 2024

(54) METHOD FOR DETERMINING A MEASURE OF RELATIVE GENE EXPRESSION

(71) Applicant: BenevolentAI Technology Limited, London (GB)

(72) Inventors: Alison McGarvey, Oxford (GB); Ana Leite, London (GB); Delphine Rolando, London (GB); Gabriel Rosser, Croydon (GB)

(21) Appl. No.: 18/326,181

(22) Filed: May 31, 2023

(51) Int. Cl. G16B 25/10; G16B 15/30

(52) U.S. Cl. CPC G16B 25/10; G16B 15/30

(57) ABSTRACT

A computer-implemented method for determining a measure of relative gene expression is disclosed. The method comprises: receiving a plurality of gene expression datasets, wherein each gene expression dataset comprises gene expression levels for a respective sample, and wherein the plurality of gene expression datasets are all measured using a first transcriptomic platform; computing a distribution of gene expression levels across the plurality of gene expression datasets; fitting a number of Gaussian components to the distribution of gene expression levels using a Gaussian mixture model; defining, based on the fitted Gaussian components, a set of relative gene expression thresholds for the plurality of gene expression datasets; and determining a measure of relative gene expression for each of a plurality of genes across the plurality of gene expression datasets based on the set of relative gene expression thresholds.

### FIELD OF THE INVENTION

The present invention relates to a computer-implemented method for determining a measure of relative gene expression. The present invention also relates to a computer-implemented method for identifying a drug target for a disease, and a computer-implemented method for assessing the safety of a drug target.

### BACKGROUND

Gene expression refers to the process in which information from a gene causes the synthesis of a functional gene product, such as a protein or non-coding RNA.

In other words, it is the process by which genotype information gives rise to the phenotype (i.e., observable characteristics).

The associated process by which a cell is able to control which genes in its DNA are expressed is known as regulation. This means that different cells in a multicellular organism may express substantially different patterns of gene expression, even though they contain identical DNA. By expressing different sets of genes, each type of cell is able to synthesise different sets of proteins, making each type of cell uniquely specialised to perform its function.

In recent years, studying gene expression has become an increasingly important component in drug discovery processes, and in particular plays a key role in the selection of potential therapeutic targets. A drug target is a biological entity in the body, such as a protein or gene, that is intrinsically related to a particular disease process and that may be addressed by a drug to produce a desired therapeutic effect with respect to the particular disease. Within drug discovery programs, the primary cause of failure is poor target selection. This results in the development of drugs which may be effective inhibitors, but which are ineffective at treating the disease of interest. By utilising bioinformatic analysis of gene expression to identify and validate therapeutic targets, the success rate of the drug discovery process can be significantly improved.

To this end, it is known that in a patient with a particular disease, certain cell types have increased or decreased gene expression in the context of that disease, relative to a healthy individual. For example, gene(s) associated with or part of the same transcriptional program as a target may show an altered expression after drug administration in response to the perturbation of the target, while other genes which are not functionally related to a target may not show an altered expression. It is also important to verify that a gene's expression level is compatible with the drug perturbation being sought. For example, small molecule antagonists inhibit the protein product of a gene, which will only be effective if the gene is present in sufficient quantity for the inhibition to result in a substantial effect on the cell being treated. Conversely, agonists activate their targets and this will only result in a meaningful perturbation against a background of low expression.

Gene expression may be measured using a variety of transcriptomic technologies, e.g., microarrays or RNA-seq. Additionally, each transcriptomic technology may be administered using different transcriptomic platforms. Technical differences in the way that these technologies and platforms quantify gene expression mean that absolute expression values cannot be assumed comparable between them.

Different platforms measure gene expression using different units across different scales and exhibit different distributions of these values. As a result, it is often not possible to assign a biological interpretation to a given absolute value measured by a single platform, corresponding to a single gene, in absence of the context of the distribution of values across all genes.

Many methods have been devised in order to normalise the gene expression levels in samples measured by a single platform, e.g., rank or percentile based approaches. However, attempts to normalise gene expression levels measured by different platforms are limited and widely accepted as being unsatisfactory. In particular, since the context of the distribution of values across all genes varies by platform, not only are absolute values not in general comparable across platforms, but even the normalised values such as rank or percentile cannot be compared.

This means that it is currently difficult to compare levels of gene expression measured by different transcriptomic technologies and platforms, such that (big data) analytics regarding gene expression is often limited to using data from one particular transcriptomic platform.

Assessing whether a candidate is expressed appropriately to be used as a therapeutic target is typically performed by a bioinformatician who categorises the relative gene expression level of each gene in the sample based on visual inspection and statistical analysis. This is a time consuming process with a tendency for inaccuracy and inconsistency, particularly when utilising data measured by a variety of transcriptomic platforms and technologies.

An object of the present invention is therefore to provide an improved method for determining a measure of relative gene expression which is more adaptable to different datasets. In particular, it is desirable to provide a method which provides accurate and comparable results across different transcriptomic technologies and platforms, i.e., irrespective of the measured type of distribution of gene expression levels.

A further object of the present invention is to enable the identification of drug targets based on gene expression analysis across one or more transcriptomic technologies and platforms.

### SUMMARY OF INVENTION

According to a first aspect of the present invention, there is provided a computer-implemented method for determining a measure of relative gene expression, comprising: receiving a plurality of gene expression datasets, wherein each gene expression dataset comprises gene expression levels for a respective sample, and wherein the plurality of gene expression datasets are all measured using a first transcriptomic platform, computing a distribution of gene expression levels across the plurality of gene expression datasets; fitting a number of Gaussian components to the distribution of gene expression levels using a Gaussian mixture model; defining, based on the fitted Gaussian components, a set of relative gene expression thresholds for the plurality of datasets; and determining a measure of relative gene expression for each of a plurality of genes across the plurality of gene expression datasets based on the set of relative gene expression thresholds.

In this way, the method is able to generate discrete (categorical) measures of gene expression from continuous gene expression values. This is achieved by first defining thresholds based on the distribution of measured expression levels across many genes in many samples, in which the thresholds are based on expression levels which have all been measured using the same transcriptomic platform. The relative measure of expression for each individual gene can then be calculated based on these thresholds. In contrast, previous attempts at discretisation have attempted to calculate thresholds based on the distribution of expression levels for a particular gene across different datasets measured using a variety of transcriptomic platforms, i.e., thresholds are computed per gene across samples. This means that if a gene does not change its expression level across the reference datasets it is not possible to calculate any thresholds. In addition, these previous techniques rely on a large reference sample size so are not adaptable to all dataset sizes. Assessing variability across a plurality of datasets also does not account for measurement biases which arise due to the use of different transcriptomic platforms to generate each dataset. The present invention overcomes these issues by, for a plurality of datasets which have been measured using the same transcriptomic platform, defining thresholds based on the overall variability of gene expression levels across the datasets. The method also ensures that biological variance is the only factor which affects the measures of relative gene expression. That is, the technical details of a platform defines the broad functional form of the distribution of gene expression levels for that platform and, within this, samples will often have similar distributions. Changes due to the sample (i.e., biological variation) are small compared to changes due to the technology (i.e., technical variation). However, the present invention is able to model the full range of biological variation (i.e., define a process that will work across a bunch of different samples) based on an understanding of the technical variation (i.e., a set of rules defining how to fit the distribution).

It will be understood that the term transcriptomic platform refers to a particular application of a type of transcriptomic technology. It will also be understood that each transcriptomic platform will produce a dataset having a particular type of distribution of gene expression levels.

The term “measure of relative gene expression” may also be referred to as a relative measure of gene expression or a discrete categorical measure.

Preferably, wherein defining the set of relative gene expression thresholds based on the fitted Gaussian components comprises: selecting a set of criteria based on the first transcriptomic platform used to measure the plurality of gene expression datasets; and evaluating the set of criteria based on the fitted Gaussian components to define the set of relative gene expression thresholds. In this way, as the thresholds are defined based on criteria associated with the transcriptomic platform used to generate the gene expression datasets, the method is able to provide relative measures of gene expression which are consistent across different types of gene expression distributions. This enables the comparison of gene expression levels between samples measured by different transcriptomic technologies and platforms. Specifically, a particular transcriptomic platform is known to produce a particular distribution of gene expression. By selecting the set of criteria for defining the threshold based on the particular transcriptomic platform, i.e., utilising a first set of criteria for a first transcriptomic platform and a second set of criteria for a second transcriptomic platform, it is possible to provide a normalised relative measure of gene expression which is independent of the transcriptomic platform used to generate the dataset.

Preferably, wherein the set of criteria are selected based on a first data structure comprising a set of transcriptomic platforms each having an associated set of criteria. For example, the first data structure may be a dictionary which specifies an applicable set of criteria for each transcriptomic platform (i.e., a respective set of criteria for each type of distribution of gene expression levels).

Preferably, wherein the number of Gaussian components is selected based on the first transcriptomic platform used to measure the plurality of gene expression datasets. In this way, the method is able to provide relative measures of gene expression which are consistent across different types of gene expression distributions. It has been found that particular types of gene expression distributions are best modelled by a particular number of Gaussian components. This is largely a technical characteristic of the transcriptomic technology and process used to measure the gene expression, including the complete experimental protocol followed to prepare an analyte from a “raw” sample and the laboratory instrumentation used to assess gene expression from the analyte. Different technical approaches vary in their sensitivity across a range of gene expression levels and their ability to detect certain genes, thereby resulting in different characteristic gene expression distributions. Additionally, research has shown that the components of gene expression distributions (e.g., bimodal, high and low expression peaks) have inherent biological meaning. Consequently, particular types of gene expression distributions are best modelled by a particular number of Gaussian components.

As the set of relative expression thresholds are determined based on the fitted Gaussian components, this means that by selecting the number of Gaussian components based on the particular transcriptomic platform, i.e., utilising a first number of Gaussian components for a first transcriptomic platform and a second number of Gaussian components for a second transcriptomic platform, it is possible to provide a consistent relative measure of gene expression which is independent of the transcriptomic platform used to generate the dataset.

Preferably, wherein the number of Gaussian components is selected based on a second data structure comprising a set of transcriptomic platforms each having an associated number of Gaussian components. For example, the second data structure may be a dictionary which specifies an optimal number of Gaussian components for each transcriptomic platform (i.e., for each type of distribution of gene expression levels). The optimal number of Gaussian components for each transcriptomic platform may by predetermined by fitting a range of Gaussian mixture models with different numbers of components to a plurality of gene expression datasets measured using a particular transcriptomic platform and determining the best fit, e.g., using a Kolmgorov-Smirnov statistic and/or by visual inspection.

It will be appreciated that a single data structure may comprise a set of transcriptomic platforms each having an associated number of Gaussian components and an associated set of criteria.

Preferably, wherein the set of criteria comprise one or more of: a crossing point between Gaussian components; a mean of a Gaussian component; and a mean of a Gaussian component plus or minus an aspect of the standard deviation of the Gaussian component.

Preferably, wherein the particular transcriptomic platform operates based on one of the following transcriptomic techniques: RNA-sequencing; or microarray analysis.

Preferably, wherein the plurality of gene expression datasets is a first plurality of gene expression datasets all measured using the first transcriptomic platform, wherein the distribution of gene expression levels across the first plurality of gene expression datasets is a first distribution of gene expression levels, and wherein the set of relative gene expression thresholds for the first plurality of gene expression datasets is a first set of relative gene expression thresholds, the method further comprising: receiving a second plurality of gene expression datasets, wherein each of the second plurality of gene expression datasets comprises gene expression levels for a respective sample, and wherein the second plurality of gene expression datasets are all measured using a second transcriptomic platform that is different to the first transcriptomic platform; computing a second distribution of gene expression levels across the second plurality of gene expression datasets; fitting a number of Gaussian components to the second distribution of expression levels using a Gaussian mixture model; defining, based on the fitted Gaussian components, a second set of relative gene expression thresholds for the second plurality of gene expression datasets, wherein the step of determining a measure of relative gene expression comprises: determining a measure of relative gene expression for each of a plurality of genes across the first and second pluralities of datasets based on the corresponding set of relative gene expression thresholds. In this way, the method enables gene expression data which has been measured by different transcriptomic platforms to be compared.

It will be appreciated that the preferably clauses above may apply equally for defining the second set of relative gene expression thresholds for the second plurality of gene expression datasets.

According to a second aspect of the present invention, there is provided a computer-implemented method for identifying a drug target for a disease, comprising: determining, using the method according to the first aspect, a first measure of relative gene expression for a gene from a first sample associated with a disease of interest; and selecting the gene or associated biological entity as a drug target for the disease of interest based on the first measure of relative gene expression.

In this way, the discrete measures of gene expression generated from continuous gene expression values are utilised in order to identify therapeutic targets for drug discovery. As a result of providing consistent measures of gene expression between different types of transcriptomic platforms, the identification of targets is not biased by the type of transcriptomic platform used to produce a particular dataset, thereby resulting in more reliable identification of drug targets.

Preferably, wherein the method further comprises: determining, using the method according to the first aspect, a second measure of relative gene expression for the same gene from a second sample; and selecting the gene or associated biological entity as the drug target for the disease of interest based on the first and second measures of relative gene expression.

Preferably, wherein the gene expression levels of the first and second samples have been measured using different transcriptomic platforms. In this way, by utilising consistent relative measures of gene expression from a plurality of datasets measured by a plurality of transcriptomic platforms, the reliability and adaptability associated with the identification of drug targets may be substantially improved.

In one example, the gene or associated biological entity is selected as a drug target for the disease of interest if it is determined that the first and/or second measure of relative gene expression corresponds to a particular measure of relative gene expression. Preferably, wherein the method further comprises: selecting a drug that incites a biological effect with respect to the drug target as a drug candidate for the disease of interest.

According to a third aspect of the invention, there is provided a method for assessing the safety of a potential drug target, comprising: receiving an indication of a gene that is differentially expressed between healthy and diseased samples of a particular cell type; determining, using the method according to the first aspect, a measure of relative gene expression for the gene from at least one healthy sample of at least one other cell type that is different to the particular cell type; and determining whether there is a safety risk associated with using the gene as a drug target based on the measure of relative gene expression.

In one example, it is determined that there is a safety risk associated with using the gene as a drug if the measure of relative gene expression is determined to correspond to a particular measure of relative gene expression.

For example, if a potential target has been identified due to its differential expression in diseased versus healthy tissues in one tissue context (e.g., in brain tissue), then the method of the first aspect may be used to check whether the potential drug target is highly expressed in any other healthy tissues (e.g., in healthy cardiac tissue). If the candidate target is found to be highly expressed in other healthy tissues, it would indicate a potential safety risk.

According to a fourth aspect of the present invention, there is provided a computer-readable medium comprising data or instruction code, which when executed on a processor, causes the processor to implement the computer-implemented method of the first, second or third aspect.

According to a fifth aspect of the invention, there is provided a computer program product comprising instructions which, when the program is executed by a computer, cause the computer to carry out the method of the first, second or third aspect.

According to a sixth aspect of the invention, there is provided a system comprising a processor configured to perform the method of the first, second or third aspect.

### BRIEF DESCRIPTION OF DRAWINGS

FIG. 1 is a flowchart showing method steps for determining a measure of relative gene expression;

FIG. 2 is a series of graphs illustrating exemplary gene expression distributions;

FIG. 3A shows an exemplary gene expression distribution having two fitted Gaussian components;

FIG. 3B shows an exemplary gene expression distribution having three fitted Gaussian components;

FIG. 4 is a flowchart showing method steps for defining a set of relative gene expression thresholds based on fitted Gaussian components;

FIG. 5A shows the exemplary gene expression distribution of FIG. 3A in which the fitted Gaussian components have been used to define relative gene expression thresholds;

FIG. 5B shows the exemplary gene expression distribution of FIG. 3B in which the fitted Gaussian components have been used to define relative gene expression thresholds;

FIG. 6 is an exemplary table illustrating calculated measures of relative gene expression for a number of genes;

FIG. 7 is a flowchart showing method steps for identifying a drug target for a disease;

FIG. 8 is a flowchart showing method steps for assessing the safety of a potential drug target; and

FIG. 9 is a block diagram of system components of a computing system in an embodiment of the invention.

### DETAILED DESCRIPTION

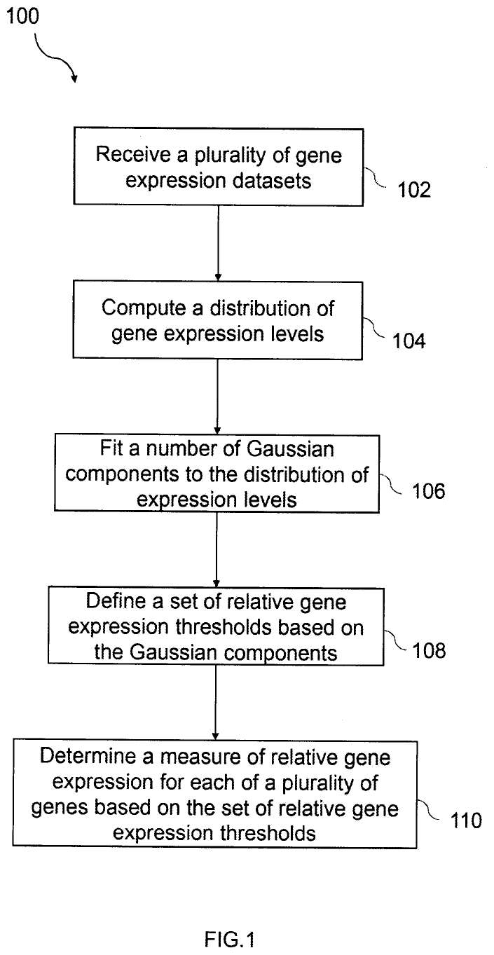

FIG. 1 illustrates a method 100 of determining a measure of relative gene expression. As used herein, the term “gene expression” will be understood to refer to the process in which information from a gene causes the synthesis of a functional gene product or a direct precursor thereof. It will be appreciated that the gene expression product itself is often not functional (e.g., messenger RNA), but it can be converted into something functional (e.g., a protein). The level of gene expression in a particular sample is quantifiable and can be measured using a variety of transcriptomic technologies, such as microarray analysis or RNA-sequencing. However, depending on the transcriptomic platform (i.e., the combination of a transcriptomic technology and a particular experimental protocol) used to measure the level of gene expression, the quantified level of gene expression can significantly differ. This means that levels of gene expression cannot be compared across different transcriptomic platforms. The method 100 disclosed herein aims to address this issue.

The method 100 begins at step 102, wherein a plurality of gene expression datasets are received. Each gene expression dataset includes data indicative of gene expression levels measured in a respective sample, i.e., a first gene expression dataset includes data indicative of gene expression levels measured in a first sample, a second gene expression dataset includes data indicative of gene expression levels measured in a second sample, and so forth.

The plurality of gene expression datasets received in step 102 are all generated using the same transcriptomic platform. In other words, the gene expression levels contained in each dataset have all been measured using the same transcriptomic platform. For example, all of the received gene expression datasets may have been measured using a particular implementation of RNA-seq (e.g., next-generation sequencing), or all of the received gene expression datasets may have been measured using a particular implementation of microarray analysis. The skilled person will understand that the measurement units for the gene expression levels will vary depending on the transcriptomic platform, e.g., RNA-sequencing typically outputs levels of gene expression in transcripts per million (TPM).

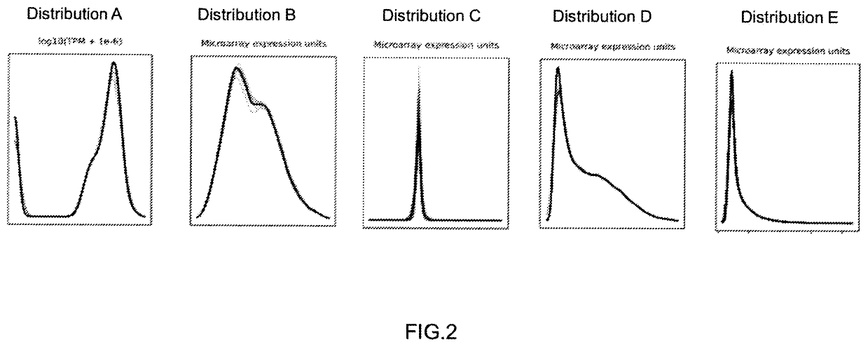

At step 104, a distribution of gene expression levels is computed across the plurality of received gene expression datasets. The computed distribution of gene expression levels represents the distribution of gene expression levels across all genes in all samples of the received datasets. This can be visualised by a density plot of gene expression levels, e.g., as illustrated in FIG. 2.

FIG. 2 shows five exemplary gene expression distribution plots. It has been found that particular transcriptomic platforms produce particular gene expression distribution shapes. This arises due to technical characteristics of the transcriptomic platform, such as their sensitivity across a range of gene expression levels and their ability to detect certain genes, in combination with various underlying biological factors. For example, distribution A in FIG. 2 illustrates a typical distribution shape for gene expression distributions measured using an RNA-sequencing transcriptomic platform, and shows a bimodal distribution due to overlapping lowly and highly expressed genes. Distributions B to E in FIG. 2 illustrate typical distribution shapes for gene expression distributions measured using four different microarray analysis transcriptomic platforms, with each distribution showing a different shape due to the specific technologies and experimental protocols implemented by each respective microarray analysis transcriptomic platform.

It is noted that a data transformation method may be applied to the computed gene expression distribution or the originally measured gene expression levels prior to computation of the gene expression distribution. For example, in the case of the gene expression datasets comprising RNA-seq gene expression data, a log transformation may be applied (e.g., a log 10 transformation). Gene expression data for RNA-seq is measured in units of TPM which typically produces a heavy-tailed distribution but, if the data is log transformed, the distribution becomes more Gaussian-like.

At step 106, a number of Gaussian components are fitted to the distribution of gene expression levels using a Gaussian mixture model. Prior to fitting, the number of Gaussian components for fitting is selected based on the transcriptomic platform used to measure the received gene expression datasets. That is, as each transcriptomic platform is associated with a particular gene expression distribution shape, the optimal number of Gaussian components for modelling distributions of gene expression for each transcriptomic platform may be predetermined.

Specifically, the optimal number of Gaussian components for each transcriptomic platform may by predetermined by fitting a range of Gaussian mixture models with different numbers of components to a plurality of gene expression datasets measured using a particular transcriptomic platform and determining the best fit, e.g., using a Kolmgorov-Smirnov statistic and/or by visual inspection. The number of Gaussian components which provides the best fit for the particular transcriptomic platform is determined to be the optimal number of Gaussian components for modelling the particular transcriptomic platform.

For example, it has been found that two Gaussian components typically provide the best fit for distribution A in FIG. 2, whereas three Gaussian components typically provide the best fit for distribution B in FIG. 2. Hence, if the received gene expression datasets have been measured using the RNA-sequencing transcriptomic platform associated with distribution A, two Gaussian components may be selected for fitting. Conversely, if the received gene expression datasets have been measured using the microarray analysis transcriptomic platform that is associated with distribution B, three Gaussian components may be selected for fitting.

To this end, a first data structure may be created comprising data indicative of a set of transcriptomic platforms each having an associated number of Gaussian components. The associated number of Gaussian components corresponds to the predetermined optimal number of Gaussian components. The number of Gaussian components to be fitted in step 106 may be selected by querying the first data structure based on the transcriptomic platform used to measure the received gene expression datasets. The skilled person will appreciate the first data structure may be referred to as a dictionary.

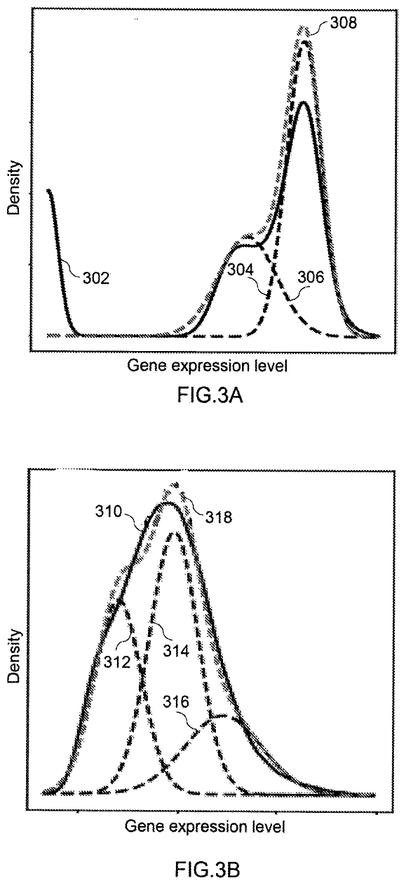

FIGS. 3A and 3B illustrate exemplary gene expression distributions having fitted Gaussian components. FIG. 3A illustrates a gene expression distribution profile 302 which has been computed based on gene expression datasets measured using an RNA-sequencing transcriptomic platform (e.g., corresponding to distribution A in FIG. 2). Based on the first data structure, it may be determined that two Gaussian components are associated with this particular transcriptomic platform. Hence, two Gaussian components 304, 306 are fitted with respect to the gene expression distribution profile 302 using a Gaussian mixture model. The sum of Gaussian components 304 and 306 is shown by line 308. FIG. 3B illustrates a gene expression distribution profile 310 which has been computed based on gene expression datasets measured using a microarray analysis transcriptomic platform (e.g., corresponding to distribution B in FIG. 2). Based on the first data structure, it may be determined that three Gaussian components are associated with this particular transcriptomic platform. Therefore, three Gaussian components 312, 314 and 318 are fitted with respect to the gene expression distribution profile 310 using a Gaussian mixture model. The sum of Gaussian components 312, 314 and 318 is shown by line 318.

Of course, it will be appreciated that there may be exceptions in which the predetermined number of Gaussian components do not optimally model the associated gene expression distribution. For example, although gene expression datasets measured using RNA-seq typically exhibit an overlapping bimodal distribution due to overlapping lowly and highly expressed genes, in certain cases the lower (leftmost) component may disappear if there are not enough lowly-expressed genes for the shoulder to appear on the distribution. In this case, the distribution may best fitted with a single Gaussian component. To address this, after fitting of the Gaussian components, it may be checked whether the distance between the peak of the first Gaussian component and the crossing point with the second Gaussian component is less than half the value of the standard deviation of the first Gaussian component. If so, the distribution may be refitted using one Gaussian component.

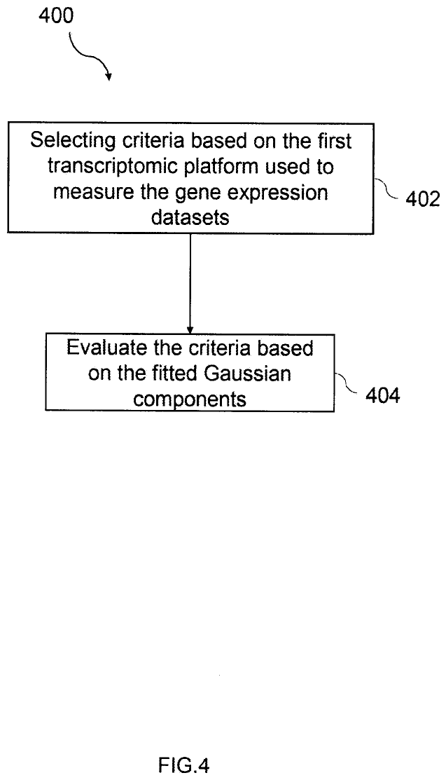

At step 108, a set of relative gene expression thresholds are defined based on the fitted Gaussian components. In other words, statistical properties of the fitted Gaussian components are used to define a set of gene expression values which define ranges of gene expression values, each range being associated with a measure of relative gene expression. The statistical properties used to determine the set of relative gene expression thresholds are chosen based on the particular transcriptomic platform used to measure the received gene expression datasets, as will be explained in further detail with reference to FIG. 4. It will be understood that the calculated set of relative gene expression thresholds apply across all the received gene expression datasets.

FIG. 4 illustrates a method 400 of defining the set of relative gene expression thresholds based on the fitted Gaussian components. At step 402, a set of criteria for defining the gene expression thresholds is selected based on the transcriptomic platform used to measure the received gene expression datasets. That is, as each transcriptomic platform is associated with a particular gene expression distribution shape, optimal criteria for defining gene expression thresholds with respect to each transcriptomic platform may be predetermined. In other words, if the received gene expression datasets are measured using a particular transcriptomic platform, a set of criteria will be selected corresponding to that particular transcriptomic platform. Examples of criteria include a crossing point between Gaussian components, a mean of a Gaussian component, and a mean of a Gaussian component plus or minus an aspect of the standard deviation of the Gaussian component (e.g., 0.5, 1, 1.5, or 2 standard deviations etc.).

The set of criteria may be determined based on an iterative process of sampling datasets measured using a particular transcriptomic platform, applying test criteria, visually inspecting the resultant gene expression thresholds, and updating the test criteria. In this regard, a key consideration is that the resultant set of relative gene thresholds should partition the subpeaks of a distribution shape in a consistent manner even where there are fluctuations in the distribution shape, such as sharper or shallower subpeaks. Another key consideration is that each threshold should be separated by a similar range of expression units. Other considerations relate to the known technological strengths and limitations of each transcriptomic technology or platform. For example, for RNA-seq datasets it is known that the low peak in a distribution of gene expression levels represents “transcriptional noise”. Therefore, criteria can be assigned such that gene expression thresholds are calculated which provide a particular measure of relative gene expression for genes falling within the low peak. The second peak in a distribution of gene expression levels for RNA-seq datasets is known to represent expressed genes so criteria can be assigned such that gene expression thresholds are calculated which provide a different measure or measures of relative gene expression for genes falling within the second peak. For microarray datasets, these distinct groups of transcriptional noise and real expression do not occur, so criteria for datasets measured using microarray transcriptomic platforms will predominantly be based on the two key considerations above.

A second data structure may be created comprising data indicative of a set of transcriptomic platforms each having an associated set of criteria for defining relative gene expression thresholds. The set of criteria may be selected in step 402 by querying the second data structure based on the transcriptomic platform used to measure the received gene expression datasets. The skilled person will appreciate that the second structure may be referred to as a dictionary. The skilled person will also appreciate that the first and second data structures may be combined to provide a single data structure comprising a set of transcriptomic platforms each having an associated number of Gaussian components and an associated set of criteria.

At step 404, the set of criteria is evaluated based on the fitted Gaussian components. For example, if a first criterion corresponding to a first relative gene expression threshold is a crossing point between first and second Gaussian components, the crossing point is determined based on the fitted Gaussian components and the first relative gene expression threshold is defined as the gene expression level at the crossing point. If a second criterion corresponding to a second relative gene expression threshold is a mean of the second Gaussian component, the mean of the second Gaussian component is determined and the second relative gene expression threshold is defined as the mean gene expression level of the second Gaussian component.

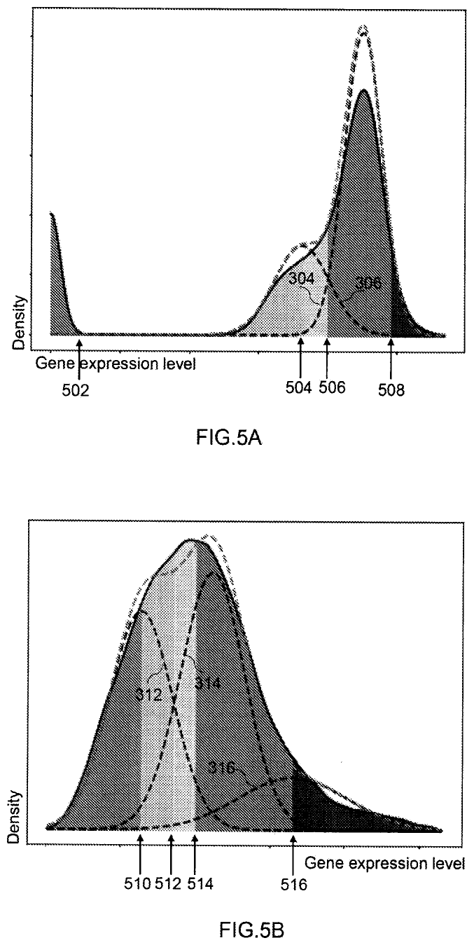

FIGS. 5A and 5B illustrate exemplary gene expression thresholds defined with respect to the gene expression distributions of FIGS. 4A and 4B respectively.

In FIG. 5A, the received gene expression datasets have been measured using a particular RNA-sequencing transcriptomic platform. Based on the second data structure, the following set of exemplary criteria are selected as being associated with the particular RNA-sequencing transcriptomic platform:

| | Relative gene expression |
|---|---|
| Criterion | threshold |
| Gene expression TPM value of | First relative gene expression |
| 0.000009 (i.e., log10(TPM + | threshold |
| 1e−6) = −5) | |
| Mean of first Gaussian component | Second relative gene expression |
| | threshold |
| Crossing point of first and second | Third relative gene expression |
| Gaussian components | threshold |
| Mean of the second Gaussian | Fourth relative gene expression |
| component + 1.5 × standard deviation | threshold |
| of second Gaussian component | |

The set of criteria are respectively evaluated to define a first relative gene expression threshold 502, a second relative gene expression threshold 504, a third relative gene expression threshold 506, and a fourth relative gene expression threshold 508 as illustrated in FIG. 5A.

In FIG. 5B, the received gene expression datasets have been measured using a particular microarray analysis transcriptomic platform. Based on the second data structure, the following set of exemplary criteria are determined as being associated with the particular microarray analysis transcriptomic platform:

| Criterion | Relative gene expression threshold |
|---|---|
| Mean of first Gaussian component | First relative gene expression |
| | threshold |
| Crossing point of first and second | Second relative gene expression |
| Gaussian components | threshold |
| Mean of second Gaussian component | Third relative gene expression |
| minus 0.5 × standard deviation of | threshold |
| second Gaussian component | |
| Mean of third Gaussian component | Fourth relative gene expression |
| | threshold |

The set of criteria are respectively evaluated to define a first relative gene expression threshold 510, a second relative gene expression threshold 512, a third relative gene expression threshold 514, and a fourth relative gene expression threshold 516 as illustrated in FIG. 5B.

At step 110, a measure of relative gene expression is assigned for one or more genes based on the set of relative gene expression thresholds. For example, each gene in each sample measured in the received gene expression datasets may be assigned a measure of relative gene expression.

The measure of relative gene expression for each gene is determined by evaluating the gene expression level of each gene with respect to the calculated gene expression thresholds. For example, a first measure of relative gene expression may be assigned to all genes which exhibit a gene expression level less than or equal to a first relative gene expression threshold. A second measure of relative gene expression may be assigned to all genes which exhibit a gene expression level greater than the first relative gene expression threshold and less than or equal to a second relative gene expression threshold. A third measure of relative gene expression may be assigned to all genes which exhibit a gene expression level greater than the second relative gene expression threshold and less than or equal to a third relative gene expression threshold, and so on. Each measure of relative gene expression may be labelled with a gene expression category, such as “not expressed”, “very low expression”, “low expression”, “medium expression”, “high expression” to aid intelligibility of the measures of relative gene expression. Advantageously, by determining from continuous gene expression values discrete measures of relative gene expression that are consistent across different transcriptomics platforms, this enables gene expression to be compared across samples that have been measured using different transcriptomic platforms.

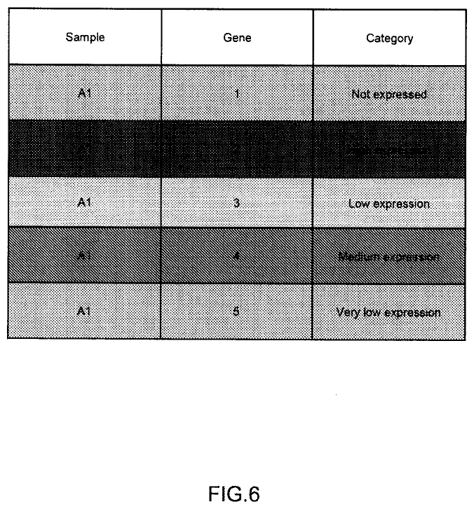

FIG. 6 is an exemplary table illustrating measures of relative gene expression which have been determined for a plurality of genes present in a sample Al based on the method 100. Each measure of relative gene expression has been labelled with corresponding gene expression categories. For example, the levels of gene expression in sample A1 may have been measured using the same RNA-sequencing transcriptomic platform that is associated with FIG. 5A. In this case, relative measures of gene expression can be determined by comparing the measured level of gene expression for each of genes 1 to 5 to the relative gene expression thresholds in FIG. 5A. Gene 1 has a gene expression level that is less than the first relative gene expression threshold 502, so gene 1 is determined to have a first measure of relative gene expression which has been labelled with the gene expression category “not expressed”. Gene 2 has a gene expression level that is more than the fourth relative gene expression threshold 508, so gene 2 is determined to have a second measure of relative gene expression which has been labelled with the gene expression category “high expression”. Gene 3 has a gene expression level that is more than the second relative gene expression threshold 504 but less than or equal to the third relative gene expression threshold 506, so gene 3 is determined to have a third measure of relative gene expression which has been labelled with the gene expression category “low expression”. Gene 4 has a gene expression level that is more than the third relative gene expression threshold 506 but less than or equal to the fourth relative gene expression threshold 508, so gene 4 is determined to have a fourth measure of relative gene expression which has been labelled with the gene expression category “medium expression”. Gene 5 has a gene expression level that is more than the first relative gene expression threshold 502 but less than or equal to the second relative gene expression threshold 504, so gene 5 is determined to have a second measure of relative gene expression which has been labelled with the gene expression category “very low expression”.

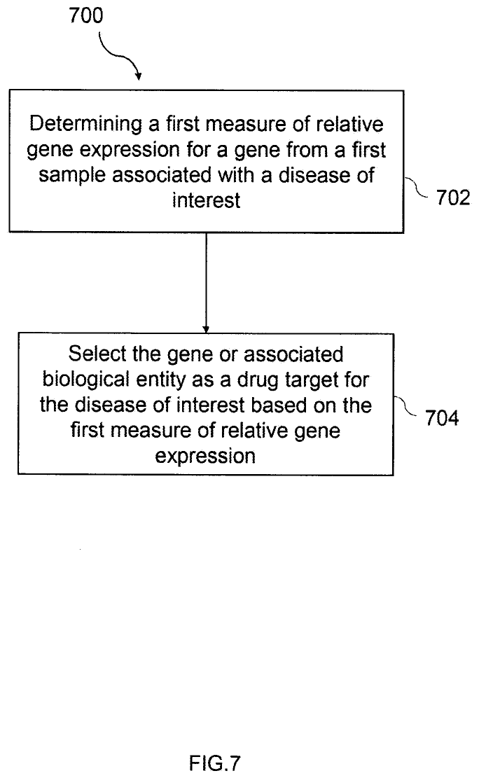

FIG. 7 illustrates a method 700 of identifying a drug target for a disease based on the method 100. At step 702, a first measure of relative gene expression is determined for a gene in a first sample associated with a disease of interest. This is achieved by receiving a measured level of gene expression for the gene in the first sample, whereby the level of gene expression has been measured using a particular transcriptomic platform. The received level of gene expression is compared to a set of relative gene expression thresholds that have been defined (based on gene expression datasets measured the same transcriptomic platform) according to the method 100, and the first measure of relative gene expression is subsequently determined.

At step 704, the gene or a biological entity associated with the gene (e.g., a functional gene product, such as a protein or non-coding RNA) is selected as a drug target for the disease of interest based on the first measure of relative gene expression. For example, the gene or associated biological entity may be selected as a drug target if the relative measure of gene expression corresponds to a particular measure of relative gene expression, e.g., a relative measure of gene expression indicative of the gene being highly expressed.

To improve the reliability of this assessment, it may be desirable to determine one or more further relative measures of gene expression for the same gene in one or more different samples. Hence, in a preferred embodiment, a second measure of relative gene expression is determined for the gene in second sample associated with the disease of interest, and the gene or associated biological entity is selected as a drug target based on the first and second measures of relative gene expression. For example, the gene or associated biological entity may be selected as a drug target if the gene has a particular measure of relative gene expression in both the first and second samples, e.g., both relative measures of gene expression are indicative of the gene being highly expressed.

It will be appreciated by the skilled person that, due to the measures of relative gene expression being standardised across different transcriptomic platforms, the levels of gene expression levels of the gene in the first and second samples may be measured using different transcriptomic platforms. This means that determination of whether to select the gene or associated biological entity as a drug target may be based on a range of gene expression data measured using a variety of transcriptomic platforms, thereby improving the reliability of the determination of a drug target.

The method 700 may further comprise selecting a drug that incites a biological effect with respect to the selected drug target as a drug candidate for the disease of interest.

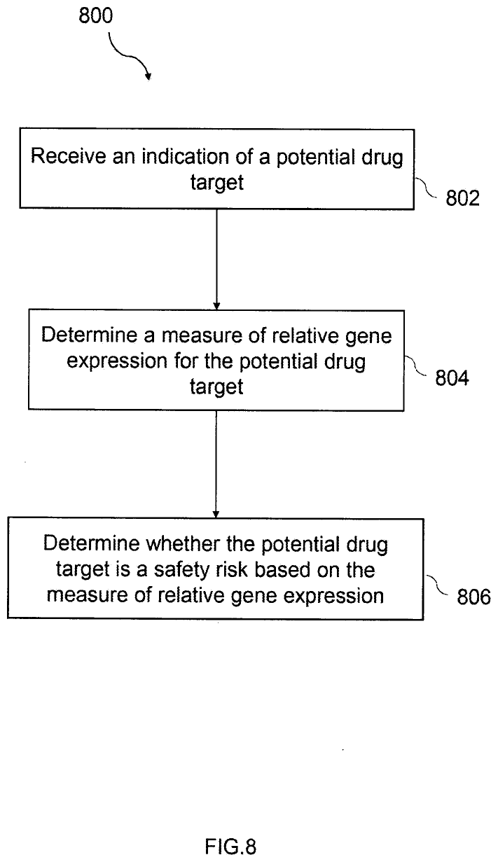

FIG. 8 illustrates a method 800 of assessing the safety of a potential drug target based on the method 100. At step 802, an indication of a potential drug target is received. The potential drug target is a gene which has been identified as being a potential drug target. For example, it may have been previously determined that the gene is differentially expressed between two samples of a particular cell type, whereby one sample is associated with a disease of interest and the other sample is not associated with the disease of interest.

At step 804, a measure of relative gene expression is determined for the gene in a further sample that is also not associated with the disease of interest, whereby the further sample corresponds to a cell type that is different to the particular cell type. That is, a gene expression level of the gene in the further sample is received and at least step 110 of method 100 is performed to determine a measure of relative gene expression for the gene in the further sample.

At step 806, it is determined whether the gene is a safety risk based on the measure of relative gene expression. For example, if the measure of relative gene expression for the gene in the further sample corresponds to a particular measure of relative gene expression (e.g., a relative measure of gene expression indicative of the gene being highly expressed), it is determined that there is a potential safety risk.

Steps 804 and 806 may be repeated to determine additional measures of relative gene expression for the gene in additional samples that are of a different cell type to the particular cell type and are not associated with the disease of interest. In this case, if any of the measures of relative gene expression are determined to have a measure of relative gene expression corresponding to a particular measure of gene expression (e.g., corresponding to high expression), the gene is determined as posing a safety risk for use a drug target. Again, the determined measures of relative gene expression may be based on data measured using different transcriptomic platforms. This means that determination of whether the gene poses a safety risk may be based on a range of gene expression data measured using a variety of transcriptomic platforms, thereby improving the reliability of the safety assessment.

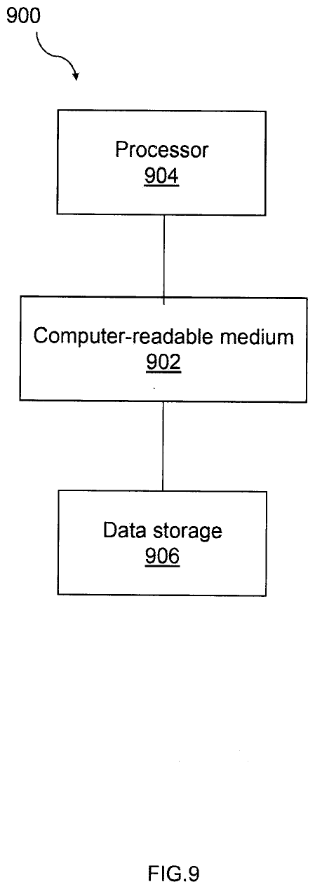

FIG. 9 is a block diagram of system components of a computing system 900. The computing system 900 comprises a computer readable medium 902 provided in electronic communication (e.g., wired or wireless) with a processor 904 and a data storage unit 906. The computer readable medium 902 may comprise memory storing a computer program having computer-executable instructions for performing the various aspects of the method disclosed above. The data storage unit 906 may contain data such as the first and second data structures.

1. A computer-implemented method for determining a measure of relative gene expression, comprising:
receiving a plurality of gene expression datasets, wherein each gene expression dataset comprises gene expression levels for a respective sample, and wherein the plurality of gene expression datasets are all measured using a first transcriptomic platform;
computing a distribution of gene expression levels across the plurality of gene expression datasets;
fitting a number of Gaussian components to the distribution of gene expression levels using a Gaussian mixture model;
defining, based on the fitted Gaussian components, a set of relative gene expression thresholds for the plurality of gene expression datasets; and
determining a measure of relative gene expression for each of a plurality of genes across the plurality of gene expression datasets based on the set of relative gene expression thresholds.

2. The computer-implemented method of claim 1, wherein defining the set of relative gene expression thresholds based on the fitted Gaussian components comprises:
selecting a set of criteria based on the first transcriptomic platform used to measure the plurality of gene expression datasets; and
evaluating the set of criteria based on the fitted Gaussian components to define the set of relative gene expression thresholds.

3. The computer-implemented method of claim 2, wherein the set of criteria are selected based on a first data structure comprising a set of transcriptomic platforms each having an associated set of criteria.

4. The computer-implemented method of claim 2, wherein the set of criteria comprise one or more of:
a crossing point between Gaussian components;
a mean of a Gaussian component; and
a mean of a Gaussian component plus or minus an aspect of the standard deviation of the Gaussian component.

5. The computer-implemented method of claim 1, wherein the number of Gaussian components is selected based on the first transcriptomic platform used to measure the plurality of gene expression datasets.

6. The computer-implemented method of claim 5, wherein the number of Gaussian components is selected based on a second data structure comprising a set of transcriptomic platforms each having an associated number of Gaussian components.

7. The computer-implemented method of claim 1, wherein the first transcriptomic platform operates based on one of:
RNA sequencing; or
microarray analysis.

8. The computer-implemented method of claim 1, wherein the plurality of gene expression datasets is a first plurality of gene expression datasets all measured using the first transcriptomic platform, wherein the distribution of gene expression levels across the first plurality of gene expression datasets is a first distribution of gene expression levels, and wherein the set of relative gene expression thresholds for the first plurality of gene expression datasets is a first set of relative gene expression thresholds, the computer-implemented method further comprising:
receiving a second plurality of gene expression datasets, wherein each of the second plurality of gene expression datasets comprises gene expression levels for a respective sample, and wherein the second plurality of gene expression datasets are all measured using a second transcriptomic platform that is different to the first transcriptomic platform;
computing a second distribution of gene expression levels across the second plurality of gene expression datasets;
fitting a number of Gaussian components to the second distribution of expression levels using a Gaussian mixture model;
defining, based on the fitted Gaussian components, a second set of relative gene expression thresholds for the second plurality of gene expression datasets,
wherein the step of determining a measure of relative gene expression comprises:
determining a measure of relative gene expression for each of a plurality of genes across the first and second pluralities of datasets based on the corresponding set of relative gene expression thresholds.

9. A computer-implemented method for identifying a drug target for a disease, comprising:
determining, using the computer-implemented method of claim 1, a first measure of relative gene expression for a gene from a first sample associated with a disease of interest; and
selecting the gene or associated biological entity as a drug target for the disease of interest based on the first measure of relative gene expression.

10. The computer-implemented method of claim 9, further comprising:
determining a second measure of relative gene expression for the same gene from a second sample; and
selecting the gene or associated biological entity as the drug target for the disease of interest based on the first and second measures of relative gene expression.

11. The computer-implemented method of claim 10, wherein the gene expression levels of the first and second samples have been measured using different transcriptomic platforms.

12. The computer-implemented method of claim 9, further comprising:
selecting a drug that incites a biological effect with respect to the drug target as a drug candidate for the disease of interest.

13. A method for assessing safety of a potential drug target, comprising:
receiving an indication of a gene that is differentially expressed between healthy and diseased samples of a particular cell type;
determining, using the computer-implemented method of claim 1, a measure of relative gene expression for the gene from at least one healthy sample of another cell type that is different to the particular cell type; and
determining whether there is a safety risk associated with using the gene as a drug target based on the measure of relative gene expression.

14. A computer-readable medium comprising data or instruction code, which when executed on a processor, causes the processor to implement the computer-implemented method of claim 1.

15. A computer program product comprising instructions which, when executed by a computer, cause the computer to carry out the method of claim 1.
